(19) United States
(12) Patent Application Publication
(10) Pub. No.: US 2022/0099672 A1
(43) Pub. Date: Mar. 31, 2022

(54) Method for Treating and/or Preventing Bacteriophage Lysis During Fermentation

(71) Applicant: Locus IP Company,LLC, Solon, OH (US)

(72) Inventors: Sean FARMER, Ft. Lauderdale, FL (US); Ken ALIBEK, Solon, OH (US)

(21) Appl. No.: 17/423,861

(22) PCT Filed: Jan. 30, 2020
(86) PCT No.: PCT/US2020/015822

§ 371 Date: Jul. 17, 2021

Related U.S. Application Data
(60) Provisional application No. 62/799,327, filed on Jan. 31, 2019.

(51) Int. Cl. G01N 33/569; C12N 1/04; C12N 1/20

(52) U.S. Cl. CPC G01N 33/56983; C12N 1/04; C12R 2001/125; C12N 2500/40; C12N 1/20

(57) ABSTRACT

The subject invention relates to enhancing for the production of microorganisms and/or their growth by-products by treating and/or preventing bacteriophage contamination of bacterial cultures. Advantageously, the methods can help reduce the likelihood of total and/or partial culture loss through the direct control of bacteriophages and/or prophages that may be present in the culture. In certain embodiments, the method comprise applying an antiviral composition comprising, for example, ribavirin, to the nutrient medium in which a microorganism is being cultivated.

### CROSS-REFERENCE TO RELATED APPLICATION

This application claims priority to U.S. Provisional Patent Application No. 62/799,327, filed Jan. 31, 2019, which is incorporated herein by reference in its entirety.

### BACKGROUND OF THE INVENTION

Cultivation of microorganisms such as bacteria, yeast and fungi is important for the production of a wide variety of useful bio-preparations. Microbes and their by-products are useful in many settings, such as oil production; agriculture; remediation of soils, water and other natural resources; mining; animal feed; waste treatment and disposal; food and beverage preparation and processing; carbon sequestration; and human health.

Two principle forms of microbe cultivation exist for growing bacteria, yeasts and fungi: submerged (liquid fermentation) and surface cultivation (solid-state fermentation (SSF)). Both cultivation methods require a nutrient medium for the growth of the microorganisms, but they are classified based on the type of substrate used during fermentation (either a liquid or a solid substrate). The nutrient medium for both types of fermentation typically includes a carbon source, a nitrogen source, salts and other appropriate additional nutrients and microelements.

In particular, SSF utilizes solid substrates, such as bran, bagasse, and paper pulp, for culturing microorganisms. Submerged fermentation, on the other hand, is typically better suited for those microbes that require high moisture. This method utilizes free flowing liquid substrates, such as molasses and nutrient broth, into which bioactive compounds are secreted by the growing microbes.

One limiting factor in commercialization of microbe-based products has been the cost per propagule density, where it is particularly expensive and unfeasible to produce and apply microbial products to large scale operations with sufficient inoculum to see the benefits.

This is partly due to the difficulties in cultivating efficacious microbial products on a large scale. One such difficulty is the loss of culture that can occur due to contamination by infectious agents. This is particularly burdensome in submerged fermentation methods, where the liquid culture medium is free-flowing, thereby facilitating movement and spreading of contaminants throughout the culture.

Sterilization and sanitation are important steps before and after any fermentation process, but infectious agents often enter a culture through, for example, air and/or water supplies, or during sampling. Bacteriophages are especially virulent contaminants in fermentation systems. These viruses infect bacteria, injecting their genome into the cell cytoplasm and replicating therein. Typically, bacteriophages are comprised of proteins that encapsulate a genome of either DNA or RNA.

Some phages follow a lytic reproductive cycle, where the host cells are destroyed after the virus has replicated, allowing for the new replicates to disperse and infect new cells. Other phages follow a lysogenic reproductive cycle, where the viral genome is integrated into the host DNA. The host cells continue replicating, along with the integrated genome (prophage). Once the prophages are activated, for example, by environmental cues, they will replicate and cause lysis of the host cells.

Infection of bacterial cultures by bacteriophages, as well as prophage induction in the host cells, are serious problems in research and biotechnological laboratories, as well as in food processing, such as dairy production. Any bacterial strain can be infected by phages or harbor one or more prophages. The propagation of phages can cause partial, or even complete, lysis of the production strains and, consequently, serious disruptions to the production process, as well as considerable economic loss.

Generally, prevention strategies, such as proper lab hygiene, sterilization, decontamination and disinfection, are necessary to avoid bacteriophage contamination; however, even the most thorough prevention practices are not indefinitely foolproof against a bacteriophage infection. Once contamination is detected, the culture is typically discarded, as well as the medium used to produce it. All equipment must be sterilized, including, for example, tanks, pipes, pipettes, shakers, benches, and other surfaces that may have come into contact with the culture.

While autoclaving is a reliable method of sterilizing certain equipment, chemical sterilization is another alternative. Ethanol, sodium hypochlorite, sodium hydroxide, ascorbic acid and a multipurpose antimicrobial product known as Vikron™ are common chemicals for pre- and post-fermentation sterilization of equipment.

Phage infections can be burdensome and costly during the production of microbe-based products, particularly when production is occurring on a large scale for industrial applications. Thus, researchers have attempted to develop methods of preventing infection without having to dispose of entire batches of culture. One method is to employ slow motion growth, or even solid state fermentation, wherein reduced rates of bacterial metabolism result in a reduced phage replication rate. Another method is to modify the genome of a particular strain of interest to be resistant to bacteriophage infection. Nonetheless, slow growth is often undesirable, for example, in large-scale commercial production settings where product turnover is important. Furthermore, genetic modification of bacteria may lead to unanticipated changes in the behavior and properties of the bacteria.

Microbes have the potential to play highly beneficial roles in, for example, the oil and agriculture industries; thus, methods are needed for preventing total loss of culture due to virulent infectious contaminants, most notably, bacteriophages.

### BRIEF SUMMARY OF THE INVENTION

The subject invention relates to the production of microbe-based products for a variety of applications. Specifically, the subject invention provides materials and methods for the efficient production of beneficial microbes, as well as for the production and use of substances, such as metabolites, derived from these microbes and the substrate in, or on, which they are produced.

In certain embodiments, this invention relates to enhancing the production of microorganisms and/or their growth by-products by treating and/or preventing bacteriophage contamination of bacterial cultures. Advantageously, the methods can help reduce the likelihood of total and/or partial culture loss through the direct control of bacteriophages and/or prophages that may be present in the culture. Furthermore, the subject methods can be employed during cultivation, without the need to discard the culture, sterilize the cultivation materials, and restart the process.

In certain embodiments, the methods comprise cultivating a bacterial strain in a nutrient medium, wherein an antiviral composition is applied to the nutrient medium. The antiviral composition can be applied to the nutrient medium prior to, or concurrently with, inoculating the medium with the bacteria, and/or at any time thereafter throughout cultivation.

In specific embodiments, the antiviral composition comprises one or more antiviral compounds that work by, for example, interfering with viral DNA or RNA synthesis and/or interfering with viral DNA or RNA polymerase activity. In one embodiment, the antiviral composition comprises a combination of more than one antiviral compound, wherein at least one of the antiviral compounds is used for treatment of RNA viruses in humans. In one embodiment, the antiviral composition comprises the antiviral compound ribavirin (also, taribavirin).

Advantageously, compared to current sterilization methods that work by controlling all microorganisms that are present, the antiviral composition can effectively control bacteriophages without harming the bacteria that are being cultivated. Thus, the methods of the subject invention can allow for treatment and/or prevention of bacteriophage lysis without disrupting the fermentation process.

The bacteria can be anaerobic, aerobic, microaerophilic, facultative anaerobes and/or obligate aerobes. In one embodiment, the bacteria are spore-forming bacteria. In one embodiment, the bacteria are Bacillus spp. bacteria, e.g., Bacillus subtilis, Bacillus licheniformis, Bacillus amyloliquefaciens or Bacillus coagulans. Other bacterial species include, for example, Rhodococcus spp., Pseudomonas spp., and Azotobacter spp.

The bacteria can be cultivated using microbial cultivation processes ranging from small to large scale. The cultivation process can be, for example, submerged cultivation, solid state fermentation (SSF), and/or modifications, hybrids or combinations thereof.

The antiviral composition can be applied to nutrient medium that is a liquid, solid, or a mixture thereof. The antiviral can be applied directly to the nutrient medium as, for example, a powder or liquid, or can be mixed with water and/or dropped into the culture in the form of a capsule or pill.

In some embodiments, the method comprises testing the culture to determine if a phage infection is present. Testing can comprise, for example, PCR, flow cytometry, indicator tests, plaque assay, or other known methods. Upon detection of a phage infection, the method can comprise additional applications of the antiviral composition to prevent total culture lysis.

The subject invention provides methods for cultivation of microorganisms and production of microbial metabolites and/or other by-products of microbial growth. In one embodiment, the subject invention provides materials and methods for the production of biomass (e.g., viable cellular material), extracellular metabolites (e.g. small molecules and proteins), residual nutrients and/or intracellular components (e.g. enzymes and other proteins).

In certain embodiments, the methods are used for producing a growth by-product of a microorganism. Accordingly, the method can further comprise extracting the growth by-product for direct use or further processing and/or purification. The growth by-product can be, for example, a biosurfactant, enzyme, biopolymer, acid, solvent, amino acid, nucleic acid, peptide, protein, lipid and/or carbohydrate. In certain embodiments, the growth by-product is a biosurfactant, such as a glycolipid or a lipopeptide.

In certain embodiments, a microbe growth facility produces fresh, high-density microorganisms and/or microbial growth by-products of interest on a desired scale. The microbe growth facility may be located at or near the site of application, or at a different location. The facility produces high-density microbe-based compositions using batch, quasi-continuous, or continuous cultivation.

### DETAILED DESCRIPTION

The subject invention relates to the production of microbe-based products for a variety of applications. Specifically, the subject invention provides materials and methods for the efficient production of beneficial microbes, as well as for the production and use of substances, such as metabolites, derived from these microbes and the substrate in, or on, which they are produced.

In certain embodiments, this invention relates to enhancing the production of microorganisms and/or their growth by-products by treating and/or preventing bacteriophage contamination of bacterial cultures. Advantageously, the methods can help reduce the likelihood of total and/or partial culture loss through the direct control of bacteriophages and/or prophages that may be present in the culture. Furthermore, the subject methods can be employed during cultivation, without the need to discard the culture, sterilize the cultivation materials, and restart the process.

### Selected Definitions

As used herein, the term “control” used in reference to a virus means killing, disabling, immobilizing, or reducing population numbers of a virus, or otherwise rendering the virus substantially incapable of replicating and causing harm to a bacterial culture.

The subject invention provides “microbe-based compositions,” which means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. In certain embodiments, the microbes are present, with medium in which they were grown, in the microbe-based composition, at, for example, a concentration of at least 1×10⁴, 1×10⁵, 1×10⁶, 1×10⁷, 1×10⁸, 1×10⁹, 1×10¹⁰, 1×10¹¹, 1×10¹² or 1×10¹³ or more cells per gram or milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise only a portion of the product of cultivation (e.g., only the growth by-products), and/or the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, such as amino acids, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of propagule) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, bioemulsifiers, biopolymers, and biosurfactants.

As used herein, the term “plurality” refers to any number or amount greater than one.

As used herein “reduction” means a negative alteration, and “increase” means a positive alteration, wherein the negative or positive alteration is at least 1%, 5%, 10%, 25%, 50%, 75%, or 100%.

As used herein, “surfactant” means a compound that lowers the surface tension (or interfacial tension) between two liquids or between a liquid and a solid. Surfactants act as, e.g., detergents, wetting agents, emulsifiers, foaming agents, and dispersants. A “biosurfactant” is a surface-active substance produced by a living cell.

As used herein, “treatment” in reference to a viral infection means the eradicating, improving, reducing, ameliorating or reversing of the infection. Treatment can include, but does not require, a complete cure, meaning treatment can also include partial eradication, improvement, reduction, amelioration or reversal.

As used herein, “prevention” means avoiding, delaying, forestalling, or minimizing the onset or progression of an occurrence or situation. Prevention can include, but does not require, absolute or complete prevention, meaning the occurrence or situation may still develop at a later time and/or with a lesser intensity or severity than it would without preventative measures. Prevention can include reducing the severity of the onset of an occurrence or situation, and/or inhibiting the progression thereof to one that is more intense or severe.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates embodiments that “consist” or “consist essentially” of the recited element(s).

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Methods

The subject invention relates to enhancing the production of microorganisms and/or their growth by-products by treating and/or preventing bacteriophage contamination of bacterial cultures. Advantageously, the methods can help reduce the likelihood of total and/or partial culture loss through the direct control of bacteriophages and/or prophages that may be present in the culture. Furthermore, the subject methods can be employed during cultivation, without the need to discard the culture, sterilize the cultivation materials, and restart the process.

In one embodiment, the subject invention provides materials and methods for producing microorganisms and/or growth by-products thereof, as well as the production of biomass (e.g., viable cellular material), extracellular metabolites (e.g. small molecules, growth by-products and proteins), residual nutrients and/or intracellular components (e.g. enzymes and other proteins).

The bacteria can be cultivated using microbial cultivation processes ranging from small to large scale. The cultivation process can be, for example, submerged cultivation, solid state fermentation (SSF), and/or modifications, hybrids or combinations thereof.

In certain embodiments, methods of cultivating a microorganism and/or producing a growth by-product of a microorganism are provided, wherein the methods comprise inoculating a nutrient medium with a microorganism, e.g., a bacterial strain. An antiviral composition is applied to the nutrient medium prior to, or concurrently with, inoculation, and/or at any time thereafter throughout cultivation. The microorganism is cultivated for an amount of time to produce a culture having a desired cell density and/or a desired concentration of growth by-products.

In certain embodiments, methods of cultivating a microorganism without contamination and/or lysis due to a bacteriophage are provided.

In some embodiments, the methods can be used for treating and/or preventing bacteriophage contamination and/or bacteriophage lysis during cultivation of a microorganism. According to the subject invention, “contamination” and/or “lysis” due to infection by bacteriophages can include any degree of contamination and/or lysis, from total loss of the culture (i.e., 100%) to partial loss of the culture (i.e., less than 100% but greater than 0%).

“Applying” can comprise pouring, spraying, spreading, pipetting, or otherwise contacting the antiviral composition with the nutrient medium in such a way that it has access to any phages present in the culture. Applying can further comprise mixing the antiviral composition into the nutrient medium to ensure uniform distribution throughout the medium. The antiviral composition can be applied to nutrient medium that is a liquid, solid, or a mixture thereof. The antiviral can be applied directly to the nutrient medium as, for example, a powder or liquid, or can be mixed with water and/or dropped into the culture in the form of, for example, a capsule or pill.

In specific embodiments, the antiviral composition comprises one or more antiviral compounds that work by, for example, interfering with viral DNA or RNA synthesis and/or interfering with viral DNA or RNA polymerase activity. In one embodiment, the antiviral composition comprises a combination of more than one antiviral compound, wherein at least one of the antiviral compounds is used for treatment of RNA viruses in humans. In one embodiment, the antiviral composition comprises the antiviral compound ribavirin (also, taribavirin).

Antiviral compounds according to the subject invention include, but are not limited to, ribavirin, valacyclovir, acyclovir, famciclovir, ganciclovir, valganciclovir, brivudin, cidofovir, fomivirsen, foscarnet, penciclovir, vidarabine, favipiravir, galidesivir, remdesivir, mericitabine, moroxydine, triazavirin, asunaprevir, boceprevir, ciluprevir, danoprevir, faldaprevir, glecaprevir, grazoprevir, narlaprevir, paritaprevir, simeprevir, sovaprevir, telaprevir, vaniprevir, vedroprevir, voxilaprevir, daclataasvir, elbasvir, ledipasvir, odalasvir, ombitasvir, pibrentasvir, ravidasvir, ruzasvir, samatasvir, velpatasvir, beclabuvir, dasabuvir, deleobuvir, filibuvir, setrobuvir, sofosbuvir, radalbuvir, uprifosbuvir, pleconaril, umifenovir, adapromione, amantadine, rimantadine, oseltamivir, zanamivir, peramivir, and laninamivir.

Advantageously, compared to current sterilization methods that work by controlling all microorganisms that are present, the antiviral composition can effectively control bacteriophages without harming the bacteria that are being cultivated. Thus, the methods of the subject invention can allow for treatment and/or prevention of bacteriophage lysis without disrupting the fermentation process.

In certain embodiments, about 0.5 g/L to about 10.0 g/L, about 1.0 g/L to about 5.0 g/L, or about 1.5 g/L to about 3.0 g/L of the antiviral composition is applied to the nutrient medium.

In certain embodiments, the method is effective for treating and/or preventing infections from bacteriophages that are lytic and/or lysogenic. In some embodiments, the bacteriophage is a species that infects bacterial hosts such as, for example, Campylobacter, Cronobacter, Escherichia, Salmonella, Lactococcus, Vibrio, Erwinia, Xanthomonas, Shigella, Staphylococcus, Streptococcus, Clostridium, Pseudomonas, Mycobacterium, Neisseria, and Bacilli. In some embodiments, the bacteriophage is a species that infects other bacterial hosts, such as those listed in this description.

In certain embodiments, the bacteriophage is a member of a viral family selected from Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttavirus, Inoviridae, Leviviridae, Microviridae, Plasmaviridae, and Tectiviridae. In certain specific embodiments, the bacteriophage is from Cystoviridae or Leviviridae.

In some embodiments, the methods can be carried out alongside other methods for preventing bacteriophage contamination and/or lysis, such as, for example, hygiene protocols, sterilization, genetic modification, and/or slowing the growth of bacteria by altering cultivation conditions and/or use of solid state fermentation.

In certain embodiments, the methods are carried out in any vessel, e.g., fermenter or cultivation reactor, for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the nutrient medium comprises a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The nutrient medium may comprise a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, isopropyl, propanol, butanol, pentanol, hexanol, isobutanol, and/or glycerol; fats and oils such as soybean oil, rice bran oil, canola oil, olive oil, corn oil, sesame oil, and/or linseed oil; etc. These carbon sources may be used independently or in a combination of two or more.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances. The substrate itself can serve as a nutrient medium, or can be mixed with a liquid nutrient medium.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride and/or sodium carbonate. These inorganic salts may be used independently or in a combination of two or more.

In some embodiments, the method for cultivation may further comprise adding additional acids and/or antimicrobials in the liquid medium before and/or during the cultivation process for protecting the culture against undesirable bacterial and/or fungal contamination.

Additionally, antifoaming agents may also be used to prevent the formation and/or accumulation of foam during cultivation.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of the liquid, and air spargers for supplying bubbles of gas to the liquid for dissolution of oxygen into the liquid.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the liquid medium may be necessary.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized.

In one embodiment, the subject invention provides methods of producing a microbial metabolite by cultivating a microbe strain of the subject invention in nutrient medium with the antiviral applied thereto, under conditions appropriate for growth and production of the metabolite; and, optionally, extracting, concentration and/or purifying the metabolite. In a specific embodiment, the metabolite is a biosurfactant. The metabolite may also be, for example, ethanol, lactic acid, beta-glucan, proteins, amino acids, peptides, metabolic intermediates, polyunsaturated fatty acids, and lipids. The metabolite content produced by the method can be, for example, at least 20%, 30%, 40%, 50%, 60%, 70%, 80%, or 90%, by weight.

The biomass content of the fermentation medium may be, for example from 5 g/l to 180 g/l or more. In one embodiment, the solids content of the medium is from 10 g/l to 150 g/l.

The microbial growth by-product produced by microorganisms of interest may be retained in the microorganisms or secreted into the growth medium. In one embodiment, the medium may contain compounds that stabilize the activity of microbial growth by-product.

The method and equipment for cultivation of microorganisms and production of the microbial by-products can be performed in a batch, quasi-continuous, or continuous processes.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a microbe-free medium or contain cells, spores, mycelia, conidia or other microbial propagules. In this manner, a quasi-continuous system is created.

Advantageously, the methods of cultivation do not require complicated equipment or high energy consumption. The microorganisms of interest can be cultivated at small or large scale on site and utilized, even being still-mixed with their media. Similarly, the microbial metabolites can also be produced at large quantities at the site of need.

Microbial Strains Grown in Accordance with the Subject Invention

The microorganisms produced according to the subject invention can be, for example, bacteria, yeasts and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In preferred embodiments, the microorganisms are bacteria, including Gram-positive and Gram-negative bacteria, as well as some archaea. The bacteria may be, spore-forming, or not. The bacteria may be motile or sessile. The bacteria may be anaerobic, aerobic, microaerophilic, facultative anaerobes and/or obligate aerobes. Bacteria species suitable for use according to the present invention include, for example, Acinetobacter spp. (e.g., A. calcoaceticus, A. venetianus); Agrobacterium spp. (e.g., A. radiobacter), Azotobacter spp. (A. vinelandii, A. chroococcum), Azospirillum spp. (e.g., A. brasiliensis), Bacillus spp. (e.g., B. amyloliquefaciens, B. firmus, B. laterosporus, B. licheniformis, B. megaterium, B. mucilaginosus, B. subtilis, B. coagulans), Chlorobiaceae spp., Dyadobacter fermenters, Frankia spp., Frateuria (e.g., F. aurantia), Klebsiella spp., Microbacterium spp. (e.g., M. laevaniformans), Pantoea spp. (e.g., P. agglomerans), Pseudomonas spp. (e.g., P. aeruginosa, P. chlororaphis, P. chlororaphis subsp. aureofaciens (Kluyver), P. putida), Rhizobium spp., Rhodospirillum spp. (e.g., R. rubrum), Sphingomonas spp. (e.g., S. paucimobilis), and/or Xanthomonas spp.

In one embodiment, the microorganism is a bacteria, such a Bacillus sp. bacteria (e.g., B. subtilis, B. licheniformis, B. firmus, B. laterosporus, B. megaterium, B. amyloliquefaciens and/or B. coagulans).

In one embodiment, the microorganism is a strain of B. subtilis, such as, for example, B. subtilis var. lotuses B1 or B2, which are effective producers of, for example, surfactin and other lipopeptide biosurfactants, as well as biopolymers. This specification incorporates by reference International Publication No. WO 2017/044953 A1 to the extent it is consistent with the teachings disclosed herein.

In preferred embodiments, these B series strains are characterized by enhanced biosurfactant production compared to wild type Bacillus subtilis strains. In certain embodiments, the Bacillus subtilis strains have increased biopolymer, solvent and/or enzyme production.

Furthermore, the B series strains can survive under high salt and anaerobic conditions better than other well-known Bacillus strains. The strains are also capable of growing under anaerobic conditions. The Bacillus subtilis B series strains can also be used for producing enzymes that degrade or metabolize oil or other petroleum products.

Other microbial strains including, for example, strains capable of accumulating significant amounts of useful metabolites, such as, for example, biosurfactants, enzymes and biopolymers, can be used in accordance with the subject invention.

### Microbe-Based Compositions

The subject methods can be used to produce compositions comprising one or more microorganisms and/or one or more growth by-products thereof. In one embodiment, the composition comprises the nutrient medium containing the microorganism and/or the metabolites produced by the microorganism and/or any residual nutrients. In some embodiments, the microbes of the composition are vegetative cells or in spore form.

The product of fermentation may be used directly without extraction or purification of growth by-products. If desired, extraction and purification can be achieved using standard extraction methods or techniques known to those skilled in the art.

In one embodiment, the growth by-product is a biosurfactant. Biosurfactants are a structurally diverse group of surface-active substances produced by microorganisms. Biosurfactants are biodegradable and can be easily and cheaply produced using selected organisms on renewable substrates. Most biosurfactant-producing organisms produce biosurfactants in response to the presence of a hydrocarbon source (e.g. oils, sugar, glycerol, etc.) in the growing media. Other media components such as concentration of iron can also affect biosurfactant production significantly.

All biosurfactants are amphiphiles. They consist of two parts: a polar (hydrophilic) moiety and non-polar (hydrophobic) group. The hydrocarbon chain of a fatty acid acts as the common lipophilic moiety of a biosurfactant molecule, whereas the hydrophilic part is formed by ester or alcohol groups of neutral lipids, by the carboxylate group of fatty acids or amino acids (or peptides), by organic acids in the case of flavolipids, or, in the case of glycolipids, by a carbohydrate.

Due to their amphiphilic structure, biosurfactants increase the surface area of hydrophobic water-insoluble substances, increase the water bioavailability of such substances, accumulate at interfaces, thus reducing interfacial tension and leading to the formation of aggregated micellar structures in solution, and change the properties of bacterial cell surfaces. The ability of biosurfactants to form pores and destabilize biological membranes permits their use as antibacterial, antifungal, and hemolytic agents.

Combined with the characteristics of low toxicity and biodegradability, biosurfactants can be useful in a variety of settings including, for example, oil and gas production; bioremediation and mining; waste disposal and treatment; animal health (e.g., livestock production and aquaculture); plant health and productivity (e.g., agriculture, horticulture, crops, pest control, forestry, turf management, and pastures); and human health (e.g., probiotics, pharmaceuticals, preservatives and cosmetics).

Biosurfactants according to the subject invention include, for example, glycolipids, lipopeptides, flavolipids, phospholipids, fatty acid esters, and high-molecular-weight polymers such as lipoproteins, lipopolysaccharide-protein complexes, and/or polysaccharide-protein-fatty acid complexes.

In one embodiment, the biosurfactants of the subject compositions include glycolipids such as rhamnolipids (RLP), sophorolipids (SLP), trehalose lipids (TL), cellobiose lipids and/or mannosylerythritol lipids (MEL).

In one embodiment, the biosurfactant is a lipopeptide biosurfactant, including, for example, iturins, surfactins, fengycins, lichenysins and/or any family member thereof. Examples of lipopeptides according to the subject invention include, but are not limited to, surfactin, lichenysin, iturin (e.g., iturin A), fengycin (e.g., fengycin A and/or B), plipastatin, polymyxin, arthrofactin, kurstakins, bacillomycin, mycosubtilin, daptomycin, chromobactomycin, glomosporin, amphisin, syringomycin and/or viscosin. In a specific embodiment, the lipopeptide is surfactin or iturin A.

In some embodiments, the biosurfactants are also useful and/or known as antibiotics. In certain embodiments, the methods can be used to produce about 1 to about 30 g/L of a biosurfactant, about 5 to about 20 g/L, or about 10 to about 15 g/L.

In some embodiments, the microbial growth by-products include other metabolites. As used herein, a “metabolite” refers to any substance produced by metabolism (e.g., a growth by-product), or a substance necessary for taking part in a particular metabolic process, for example, enzymes, enzyme inhibitors, biopolymers, acids, solvents, gases, proteins, peptides, amino acids, alcohols, pigments, pheromones, hormones, lipids, ectotoxins, endotoxins, exotoxins, carbohydrates, antibiotics, anti-fungals, anti-virals and/or other bioactive compounds. The metabolite content produced by the method can be, for example, at least 0.1%, 1%, 5%, 10%, 20%, 30%, 40%, 50%, 60%, 70%, 80%, or 90% by weight.

In one embodiment, the growth by-product is a biopolymer, such as, for example, levan, xanthan gum, alginate, hyaluronic acid, PGAs, PHAs, cellulose, and lignin.

In one embodiment, the growth by-product is a bioemulsifier, such as, for example, emulsan, alasan, or liposan.

In one embodiment, the growth by-product is a protein, a lipid, a carbon source, an amino acid, a mineral or a vitamin.

In one embodiment, the growth by-products are enzymes such as, for example, oxidoreductases, transferases, hydrolases, lyases, isomerases and/or ligases. Specific types and/or subclasses of enzymes according to the subject invention can also include, but are not limited to, nitrogenases, proteases, flavodoxins, amylases, glycosidases, cellulases, glucosidases, glucanases, galactosidases, moannosidases, sucrases, dextranases, hydrolases, methyltransferases, phosphorylases, dehydrogenases (e.g., glucose dehydrogenase, alcohol dehydrogenase), oxygenases (e.g., alkane oxygenases, methane monooxygenases, dioxygenases), hydroxylases (e.g., alkane hydroxylase), esterases, lipases, ligninases, mannanases, oxidases, laccases, tyrosinases, cytochrome P450 enzymes, peroxidases (e.g., chloroperoxidase and other haloperoxidases), and lactases.

In one embodiment, the growth by-products include antibiotic compounds, such as, for example, aminoglycosides, amylocyclicin, bacitracin, bacillaene, bacilysin, bacilysocin, corallopyronin A, difficidin, etnangien gramicidin, β-lactams, licheniformin, macrolactinsublancin, oxydifficidin, plantazolicin, ripostatin, spectinomycin, subtilin, tyrocidine, and/or zwittermicin A. In some embodiments, an antibiotic can also be a type of biosurfactant.

In one embodiment, the growth by-products include anti-fungal compounds, such as, for example, fengycin, surfactin, haliangicin, mycobacillin, mycosubtilin, and/or bacillomycin. In some embodiments, an anti-fungal can also be a type of biosurfactant.

In one embodiment, the growth by-products include other bioactive compounds, such as, for example, butanol, ethanol, acetate, ethyl acetate, lactate, acetoin, benzoic acid, 2,3-butanediol, beta-glucan, indole-3-acetic acid (IAA), lovastatin, aurachin, kanosamine, reseoflavin, terpentecin, pentalenolactone, thuringiensin (β-exotoxin), polyketides (PKs), terpenes, terpenoids, phenyl-propanoids, alkaloids, siderophores, as well as ribosomally and non-ribosomally synthesized peptides, to name a few.

In certain other embodiments, the compositions comprise one or more microbial growth by-products, wherein the growth by-products have been extracted from the culture and, optionally, purified.

### Methods of Use

The compositions of the subject invention can be used for a variety of purposes. In one embodiment, the subject compositions can be highly advantageous in the context of the oil and gas industry. When applied to an oil well, wellbore, subterranean formation, or to equipment used for recovery oil and/or gas, the subject composition can be used in methods for enhancement of crude oil recovery; reduction of oil viscosity; removal and dispersal of paraffin from rods, tubing, liners, and pumps; prevention of equipment corrosion; recovery of oil from oil sands and stripper wells; enhancement of fracking operations as fracturing fluids; reduction of H₂S concentration in formations and crude oil; and cleaning of tanks, flowlines and pipelines.

In one embodiment, the composition can be used to improve one or more properties of oil. For example, methods are provided wherein the composition is applied to oil or to an oil-bearing formation in order to reduce the viscosity of the oil, convert the oil from sour to sweet oil, and/or to upgrade the oil from heavy crude into lighter fractions.

In one embodiment, the composition can be used to clean industrial equipment. For example, methods are provided wherein the composition is applied to oil production equipment such as an oil well rod, tubing and/or casing, to remove heavy hydrocarbons, paraffins, asphaltenes, scales and other contaminants from the equipment. The composition can also be applied to equipment used in other industries, for example, food processing and preparation, agriculture, paper milling, waste treatment, and others where scales, heavy hydrocarbons, fats, oils and/or greases build up and contaminate and/or foul the equipment.

In one embodiment, the composition can be used in agriculture. For example, methods are provided wherein the composition is applied to a plant and/or its environment to treat and/or prevent the spread of pests and/or diseases. The composition can also be useful for enhancing water dispersal and absorption in the soil, as well as to enhance nutrient absorption from the soil through plant roots, facilitate plant health, increase yields, and manage soil aeration.

In one embodiment, the composition can be used to enhance animal health. For example, methods are provided wherein the composition can be applied to animal feed or water, or mixed with the feed or water, and used to prevent the spread of disease in livestock and aquaculture operations, reduce the need for antibiotic use in large quantities, as well as to provide supplemental proteins and other nutrients.

In one embodiment, the composition can be used to prevent spoilage of food, prolong the consumable life of food, and/or to prevent food-borne illnesses. For example, methods are provided wherein the composition can be applied to a food product, such as fresh produce, baked goods, meats, and post-harvest grains, to prevent undesirable microbial growth.

In one embodiment, the composition can be used to enhance human and/or animal health, for example, as a probiotic, a health supplement, or as a pharmaceutical drug for treating bacterial, fungal, and/or viral infection, and/or to treat other conditions including cancers, neurodegenerative diseases, immune system conditions, digestive maladies, cardiopulmonary conditions, diabetes, neurodevelopmental diseases, and many others.

Other uses for the subject compositions include, but are not limited to, biofertilizers, biopesticides, bioleaching, bioremediation of soil and water, wastewater treatment, nutraceuticals and supplements, cosmetic products, detergents, disinfectants, and many others.

### Preparation of Microbe-Based Products

One microbe-based product of the subject invention is simply the nutrient medium containing the microorganism and/or the microbial metabolites produced by the microorganism and/or any residual nutrients. Upon harvesting of the medium, microbe, and/or by-products, the product can be homogenized, and optionally, mixed with water, e.g., in a storage tank. In some embodiments, prior to mixing with water, the product can be dried using, for example, spray drying or lyophilization. The dried product can also be stored.

The product of fermentation may be used directly without extraction or purification. If desired, extraction and purification can be achieved using standard extraction methods or techniques known to those skilled in the art.

The microorganisms in the microbe-based product may be in an active or inactive form. In some embodiments, the microorganisms have sporulated or are in spore form. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

In one embodiment, the microbe-based product can comprise at least 1×10⁴ to 1×10¹², 1×10⁵ to 1×10¹¹ or 1×10⁶ to 1×10¹⁰ cells or spores per ml. In certain preferred embodiments, the product comprises at least 1×10¹⁰ cells or spores per ml.

The dried and/or liquid product can be transferred to the site of application via, for example, tanker for immediate use. Additional nutrients and additives can be included as well.

In other embodiments, the composition (in the form of a dried product or in liquid form) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In certain embodiments the containers are 2 gallons, 5 gallons, 25 gallons, or larger.

Upon harvesting the microbe-based composition from the reactors, further components can be added as the harvested product is processed and/or placed into containers for storage and/or transport. The additives can be, for example, buffers, carriers, other microbe-based compositions produced at the same or different facility, viscosity modifiers, preservatives, nutrients for microbe growth, tracking agents, pesticides, and other ingredients specific for an intended use.

Advantageously, in accordance with the subject invention, the microbe-based product may comprise the substrate in which the microbes were grown. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., an oil well). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules (e.g., spores), or a mixture of vegetative cells and propagules.

In one embodiment, the microbe growth facility is located on, or near, a site where the microbe-based products will be used, for example, within 300 miles, 200 miles, or even within 100 miles. Advantageously, this allows for the compositions to be tailored for use at a specified location. The formula and potency of microbe-based compositions can be customized for a specific application and in accordance with the local conditions at the time of application.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

What is claimed:

1. A method of cultivating a bacterial strain and/or producing a growth by-product of a bacterial strain, the method comprising:
a) inoculating a nutrient medium with the strain;
b) applying an antiviral composition to the nutrient medium; and
c) cultivating the strain to produce a culture having a desired cell density and/or a desired concentration of the growth by-product,
wherein the antiviral composition comprises one or more antiviral compounds that interfere with viral DNA or RNA synthesis and/or interfere with viral DNA or RNA polymerase activity, and
wherein the antiviral composition controls a bacteriophage in the culture but does not harm the bacterial strain.

2. The method of claim 1, wherein the antiviral composition comprises ribavirin.

3. The method of claim 1, wherein the bacteria is a strain of Bacillus, Azotobacter, Pseudomonas or Rhodococcus.

4. The method of claim 1, wherein the nutrient medium is solid or liquid.

5. The method of claim 1, wherein the nutrient medium comprises sources of nitrogen and carbon.

6. The method of claim 1, wherein about 0.5 g/L to about 10.0 g/L of the antiviral composition is applied to the nutrient medium.

7. The method of claim 1, wherein the antiviral composition is applied prior to, or concurrently with, inoculating the nutrient medium.

8. The method of claim 1, wherein the antiviral composition is applied after inoculating the nutrient medium.

9. The method of claim 1, further comprising testing the culture for the presence of a bacteriophage infection.

10. The method of claim 9, wherein, if a bacteriophage infection is detected, the method further comprises applying one or more additional applications of the antiviral composition.

11. The method of claim 1, wherein the antiviral composition comprises one or more of ribavirin, valacyclovir, acyclovir, famciclovir, ganciclovir, valganciclovir, brivudin, cidofovir, fomivirsen, foscarnet, penciclovir, vidarabine, favipiravir, galidesivir, remdesivir, mericitabine, moroxydine, triazavirin, asunaprevir, boceprevir, ciluprevir, danoprevir, faldaprevir, glecaprevir, grazoprevir, narlaprevir, paritaprevir, simeprevir, sovaprevir, telaprevir, vaniprevir, vedroprevir, voxilaprevir, daclataasvir, elbasvir, ledipasvir, odalasvir, ombitasvir, pibrentasvir, ravidasvir, ruzasvir, samatasvir, velpatasvir, beclabuvir, dasabuvir, deleobuvir, filibuvir, setrobuvir, sofosbuvir, radalbuvir, uprifosbuvir, pleconaril, umifenovir, adapromione, amantadine, rimantadine, oseltamivir, zanamivir, peramivir, and laninamivir.

12. The method of claim 1, wherein the bacteriophage is from Cystoviridae or Leviviridae.
